(19) United States
(12) Patent Application Publication
(10) Pub. No.: US 2026/0335586 A1
(43) Pub. Date: Oct. 29, 2026

(54) HUMIDIFICATION SYSTEMS FOR SURGICAL PROCEDURES

(71) Applicant: Conmed Corporation, Largo, FL (US)

(72) Inventor: Steven Lathers, Littleton, CO (US)

(73) Assignee: Conmed Corporation, Largo, FL (US)

(21) Appl. No.: 19/103,593

(22) PCT Filed: Aug. 17, 2023
(86) PCT No.: PCT/US2023/030512

§ 371 Date: Feb. 13, 2025

Related U.S. Application Data
(60) Provisional application No. 63/399,195, filed on Aug. 18, 2022.

(51) Int. Cl. A61M 16/16

(52) U.S. Cl. CPC A61M 16/16; A61M 2205/7545

(57) ABSTRACT

In accordance with at least one aspect of this disclosure, a system for introducing humidified gas to a surgical access device (114) includes, a humidification device (202) including a housing (204) defining an interior humidification chamber (224). The 2024/039805 interior humidification chamber includes, a first inlet (226) for receiving a flow of gas, a second inlet (228) for receiving a flow of liquid, a humidification mechanism (232) configured to convert at least a portion of the flow of liquid to a flow of vapor for mixing with the flow of gas to form a flow of humidified gas, and an outlet (230) for delivering the flow of humidified gas to the surgical access device. WO

### CROSS-REFERENCE TO RELATED APPLICATIONS

This application claims the benefit of U.S. Provisional Patent Application No. 63/399,195, filed Aug. 18, 2022, the entire content of which is incorporated herein by reference.

### TECHNICAL FIELD

The present application relates to surgical procedures, and more particularly, to devices for adding humidity to a surgical cavity during a surgical procedure.

### BACKGROUND

From the beginning of laparoscopic surgical procedures some thirty years ago, it has been assumed that the condition of gases used to inflate body cavities and spaces were physiologically and pathologically benign. While the importance and use of temperature and moisture conditioning of anesthesia gases has been well known, until recently little attention had been given to the particulate, temperature and/or humidity condition of insufflation gases used to create a pneumoperitoneum. Reduction in core body temperature, introduction of foreign bodies and drying of surfaces (including peritoneal surfaces), resulting from the introduction of insufflation gases in such surgical procedures are continuing problems.

A commonly used insufflation gas is carbon dioxide which is typically provided as a liquid in compressed gas cylinders. The pressure in these cylinders, when at equilibrium with ambient environment of 200 C, is 57 atmospheres (5740 kPa). The carbon dioxide gas is typically provided to the surgical site at a pressure of 15 mmHg via an adjustable, throttling pressure regulator and flow controller called an insufflator. Many models of insufflators are available such as the Storz Model 26012 (Karl Storz Endoscopy-America The., Culver City, Calif.). In general, insufflators do not filter and few have the capability to control the gas temperature and/or humidifying the gas.

It is known to filter insufflation gas to prevent inorganic particles such as metallic fillings or particles, rust, dust, and polymer particles from passing into the pneumoperitoneum (see, e.g., Ott, D. E., j Gynecol Surg., 5:205-208 (1989)). The location and type of filter, however, are very important factors which will influence the effectiveness of the method. Filters having a pore size as small as 0.2 microns have been used in previous insufflation systems. These devices, however, utilize a filter material that is typically hydrophilic and when it becomes moist, loses its strength and some of its filtering effectiveness. Moreover, because these prior art filter devices are not hydrophobic, they lose their filtering capability by tearing under the water pressure caused by accidentally suctioning or syphoning peritoneal or irrigation fluids.

In addition, in order to compensate for the cool temperature and dryness of the carbon dioxide insufflation gas, an apparatus and method have been developed to control the temperature and humidity of the insufflation gas as it is delivered into the body. Such an apparatus and method are disclosed in commonly assigned U.S. Pat. No. 5,411,474 to Ott et al., the entirety of which is herein incorporated by reference. Nevertheless, there is room for improvement of a heating, hydrating and filtering apparatus for the delivery of insufflation gases.

### SUMMARY

In accordance with at least one aspect of this disclosure, a system for introducing humidified gas to a surgical access device includes, a humidification device including a housing defining an interior humidification chamber. The interior humidification chamber includes, a first inlet for receiving a flow of gas, a second inlet for receiving a flow of liquid, a humidification mechanism configured to convert at least a portion of the flow of liquid to a flow of vapor for mixing with the flow of gas to form a flow of humidified gas, and an outlet for delivering the flow of humidified gas to the surgical access device.

The humidification mechanism is configured and adapted to disturb the flow of liquid within the interior humidification chamber to convert the flow of liquid to the flow of vapor for mixing with the flow of gas. In embodiments, the humidification mechanism is a nebulizer. In certain embodiments, the nebulizer can be or include a vibrating mesh nebulizer or an ultrasonic nebulizer.

In certain embodiments, in the interior humidification chamber, the first inlet communicates with a branched flow path formed within the housing having a first branch and a second branch. The branched flow path extends around a periphery of the interior humidification chamber, and bypassing the interior humidification chamber, such that each of the first branch and the second branch join the outlet separately and both downstream of the interior humidification chamber. In such embodiments, the flow of gas and the flow of vapor are permitted to mix in the outlet before being delivered to the surgical access device.

In certain embodiments, in the interior humidification chamber, the first inlet communicates with a first flow path formed within the housing. The first flow path bypasses the interior humidification chamber, and the second inlet communicates directly with the interior humidification chamber within the housing. The first flow path and the second flow path meet at a T-junction upstream of the outlet, and the flow of gas and the flow of vapor mix in the T-junction, downstream of the interior humidification chamber.

The housing further includes a first inlet fitting, a second inlet fitting and an outlet fitting. The system includes, a first lumen configured to connect to the first inlet fitting to deliver the flow of gas to the interior humidification chamber from a gas source, a second lumen configured to connect to the second inlet fitting to deliver the flow of liquid to the interior humidification chamber from a liquid source, and a third lumen configured to connect to the outlet fitting to deliver the flow of humidified gas from the interior humidification chamber to the surgical access device. The first lumen connects to a gas source and the first inlet fitting, the second lumen connects to a liquid source and the second inlet fitting, and the third lumen connect to the outlet fitting and an inlet fitting of the surgical access device

In embodiments, the system further includes, a tube set for use with a surgical gas delivery device. The tube set includes, a filter cartridge having a housing defining a inlet flow path for receiving a flow of gas from a patient's abdominal cavity and an outlet flow path for delivering a flow of gas to the patient's abdominal cavity. A first lumen extends from a front end of the filter cartridge and communicates with the outlet flow path of the filter cartridge. The humidification device is an in-line humidification device within the first lumen. The in-line humidification device includes an inlet fitting and an outlet fitting, the inlet fitting communicating with a distal end of the first lumen. A second lumen extends from the outlet fitting of the in-line humidification device and communicates with the inlet flow path of the filter cartridge. The second lumen has a first connector at a distal end thereof for connecting to a surgical access device. A third lumen has a second connector at a distal end thereof for connecting to the surgical access device and extending from the surgical access device and communicates with the inlet flow path of the filter cartridge.

The second lumen is configured to deliver a mixture of fluid vapor and surgical gas to a surgical cavity to increase a relative humidity of the surgical cavity, and the third lumen is configured to deliver evacuated smoke from the surgical cavity to the cartridge. In embodiments, the system can further include the gas delivery system.

In embodiments, the inlet flow path of the filter cartridge and/or the outlet path of the filter cartridge can include one or more filter elements therein. A fourth lumen extends from the front end of the filter cartridge for providing a jet flow to the surgical access device to create an air seal at the surgical access device to prevent egress of gas from the patient's abdominal cavity through the surgical access device.

In embodiments, the fourth lumen includes a third connector at a distal end thereof for connecting to the surgical access device. In certain embodiments, the second lumen, the third lumen, and the fourth lumens can be coupled at distal ends thereof such that the first connector, the second connector, and the third connector define a common connector having respective flow paths therethrough. In certain embodiments, the common connector can be a tri-lumen connector.

In accordance with at least one aspect of this disclosure, a system, includes a surgical access device configured to deliver humidity to a surgical cavity to increase a relative humidity of the surgical cavity, a reservoir configured to retain at least a liquid therein, a humidification mechanism operatively connected to at least the reservoir configured and adapted to disturb the liquid in the reservoir to convert at least a portion of the liquid to a vapor state to produce humidity within the reservoir, a first lumen fluidly connected between the reservoir and the surgical access device to provide the vapor to the surgical access device to deliver the humidity to the surgical cavity through the surgical access device. In embodiments, the liquid can be an aqueous solution, and in certain embodiments, the aqueous solution can be a saline solution.

In embodiments, the humidification mechanism can include a heating element in thermal communication with the reservoir configured and adapted to vaporize the liquid within the reservoir to produce the vapor. An inlet of the first lumen is in fluid communication with the vapor in the reservoir. In embodiments, a second lumen can be fluid communication with a gas source (e.g., CO2) configured to deliver a flow of pressurized gas to the first lumen to provide a back pressure on the humidification mechanism. Here, a portion of the second lumen is in fluid communication with the reservoir for delivering the flow of pressurized gas to the reservoir. The second lumen includes a bypass portion in fluid communication with the first lumen downstream of the reservoir and upstream of the surgical access device.

In certain embodiments, the humidification mechanism includes nebulizer disposed at an inlet of the first lumen configured and adapted to convert the liquid at an outlet of the reservoir and the inlet of the first lumen. In embodiments, the nebulizer can be or include a vibrating mesh nebulizer, or an ultrasonic nebulizer. In embodiments, a second lumen can be in fluid communication with a gas source configured to deliver a flow of pressurized gas to the first lumen to provide a back pressure on the humidification mechanism. Here, the second lumen can intersect the first lumen downstream of the reservoir and upstream of the surgical access device. In embodiments, a heating element is in thermal communication with the reservoir configured to heat the liquid within the reservoir such that a temperature of the humidity delivered to the surgical cavity is greater than a temperature of the pressurized gas in the second lumen.

In certain embodiments, a pump is disposed in the reservoir configured and adapted to pump the liquid to the surgical access device at a pressure, and the humidification mechanism is operatively connected to at least the surgical access device configured to convert the pressurized liquid a vapor to produce the humidity. The humidification mechanism can be or include a nebulizer disposed between an outlet of the first lumen and within an inlet of the surgical access device configured and adapted to vaporize the pressurized liquid in the first lumen within the inlet of the surgical access device. In certain embodiments, a second lumen can be in fluid communication with a gas source configured to deliver a flow of pressurized gas to the first lumen to provide a back pressure on the humidification mechanism. Here, an outlet of the second lumen can intersect at the surgical access device downstream of an outlet of the first lumen and downstream of the humidification mechanism. In embodiments, a heating element is in thermal communication with the reservoir configured to heat the liquid within the reservoir such that a temperature of the humidity delivered to the surgical cavity is greater than a temperature of the pressurized gas in the second lumen

In embodiments, the surgical access device is a first surgical access device, and the system can include a surgical gas delivery system configured and adapted to deliver a flow of insufflation gas to the surgical cavity through a second surgical access device (e.g., similar to that described above). In certain embodiments, the surgical access device can be a single surgical access device for delivering humidity and surgical gas, where the surgical gas delivery system configured and adapted to deliver the flow of gas to the surgical cavity through the surgical access device. The surgical gas can include insufflation gas (e.g., CO2).

In embodiments, the surgical access device can include a multi-lumen connector configured to interface with a connector of the first lumen and a connector of a lumen of the surgical gas delivery system.

In accordance with at least one aspect of this disclosure, a system for delivering humidity to a surgical cavity includes, a surgical access device, a reservoir configured to retain at least a liquid therein, a first humidification mechanism operatively connected to the reservoir configured and adapted to disturb the liquid in the reservoir to convert at least a portion of the liquid to a vapor state to produce humidity within the reservoir, a lumen fluidly connected between the reservoir and the surgical access device to provide the vapor to the surgical access device, and a second humidification mechanism operatively connected to the surgical access device configured and adapted to disturb any remaining liquid in the lumen and/or an inlet of the surgical access device to convert the remaining liquid to a vapor state to produce humidity within the surgical access device, to deliver the humidity to the surgical cavity through the surgical access device.

In accordance with at least one aspect of this disclosure, a filtered tube set for use with a surgical gas delivery device includes, a filter cartridge having a housing defining an inlet flow path for receiving a flow of gas from a patient's abdominal cavity and an outlet flow path for delivering a flow of gas to the patient's abdominal cavity. A first lumen extends from a front end of the cartridge and communicates with the outlet flow path of the filter cartridge. A humidification device is disposed in the first lumen as an in-line humidification device (e.g., any embodiment of a humidification device described herein), the in-line humidification device including an inlet fitting and an outlet fitting, the inlet fitting communicates with a distal end of the first lumen. A second lumen extends from the outlet fitting of the in-line humidification device and communicating with the inlet flow path. The second lumen has a first connector at a distal end thereof for connecting to a surgical access device; and a third lumen having a second connector at a distal end thereof for connecting to the surgical access device extends from the surgical access device and communicating with the inlet flow path of the filter cartridge. In embodiments, the filter cartridge is configured to connect to the surgical gas delivery system.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is an illustration of a surgical arena during a laparoscopic procedure performed on a patient utilizing a multi-modal gas delivery device and an associated filtered tube set which includes a humidification system constructed in accordance with an exemplary embodiment of the subject invention;

FIG. 2 is a perspective view of an embodiment of a filtered tube set configured for interfacing with the gas delivery device of FIG. 1, which includes a filter cartridge having a tube set attached thereto and an embodiment of an in-line humidification device;

FIG. 3A is a side elevation view of the in-line humidification device of FIG. 2;

FIG. 3B is a cross-sectional view of the humidification device of FIG. 3A shown connected to a tube set taken along line 3B-3B;

FIG. 4 shows the tube set of FIG. 3A connected to a surgical access device where the humidification device is shown in cross-section;

FIG. 5 is a perspective view of an embodiment of a filtered tube set configured for interfacing with the gas delivery device of FIG. 1, which includes a filter cartridge having a tube set attached thereto and an embodiment of an in-line humidification device;

FIG. 6A is a side elevation view of an embodiment of the in-line humidification device of FIG. 5;

FIG. 6B is a cross-sectional view of the humidification device of FIG. 6A shown connected to a tube set taken along line 6B-6B;

FIG. 7 shows the tube set of FIG. 6A connected to a surgical access device where the humidification device is shown in cross-section;

FIG. 8A is side view of an embodiment of a humidification system having a first lumen connecting between a reservoir and a surgical access device and a humidification device connected to the reservoir, where a wall of the first lumen and a wall of the reservoir are broken away to review the flow therein;

FIG. 8B is side view of an embodiment of a humidification system having a first lumen connecting between a reservoir and a surgical access device and a second lumen connecting between a fluid source and the first lumen and between the reservoir and the first lumen, where a wall of the first lumen, a wall of the second lumen, and a wall of the reservoir are broken away to review the flow therein;

FIG. 9A is side view of an embodiment of a humidification system having a first lumen connecting between a reservoir and a surgical access device and a humidification device connected to an outlet of the reservoir and an inlet of the first lumen, where a wall of the first lumen and a wall of the reservoir are broken away to review the flow therein;

FIG. 9B is side view of an embodiment of a humidification system having a first lumen connecting between a reservoir and a surgical access device and a second lumen connecting between a fluid source and the first lumen, where a wall of the first lumen, a wall of the second lumen, and a wall of the reservoir are broken away to review the flow therein; FIG. 10A is side view of an embodiment of a humidification system having a first lumen connecting between a reservoir and a surgical access device and a humidification device within a surgical access device, where a wall of the first lumen and a wall of the reservoir are broken away to review the flow therein;

FIG. 10B is side view of an embodiment of a humidification system having a first lumen connecting between a reservoir and a surgical access device and a second lumen connecting between a fluid source and a surgical access device, where a wall of the first lumen, a wall of the second lumen, and a wall of the reservoir are broken away to review the flow therein;

### DETAILED DESCRIPTION

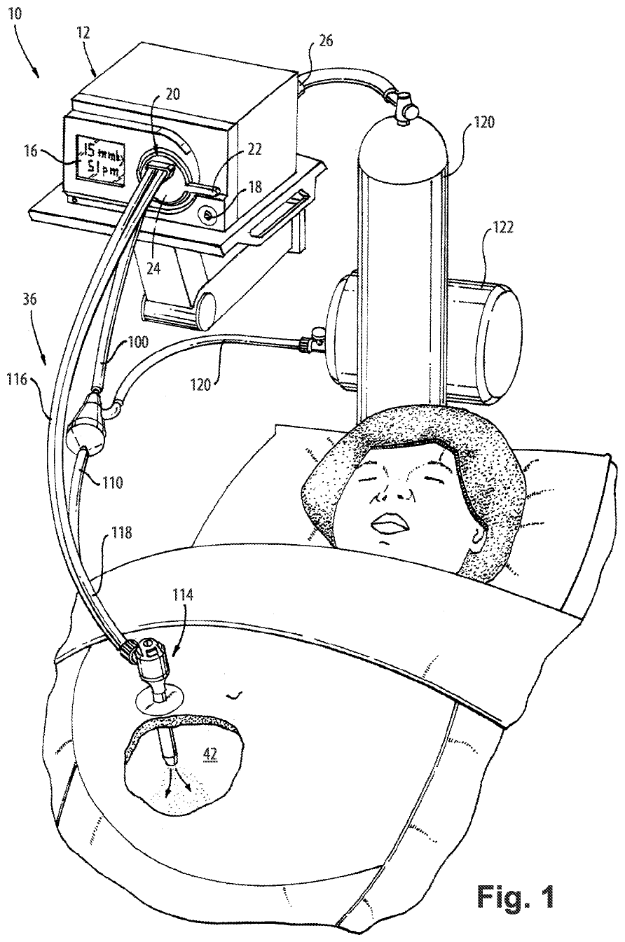

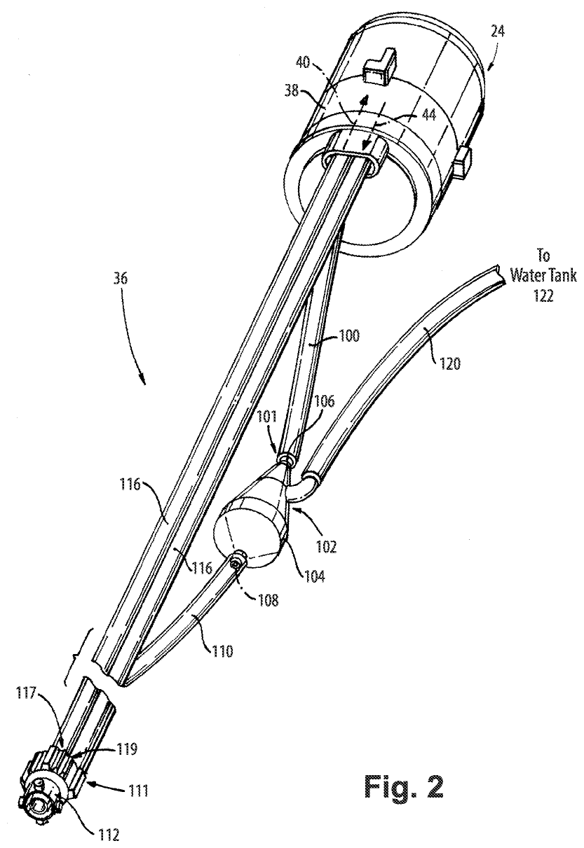

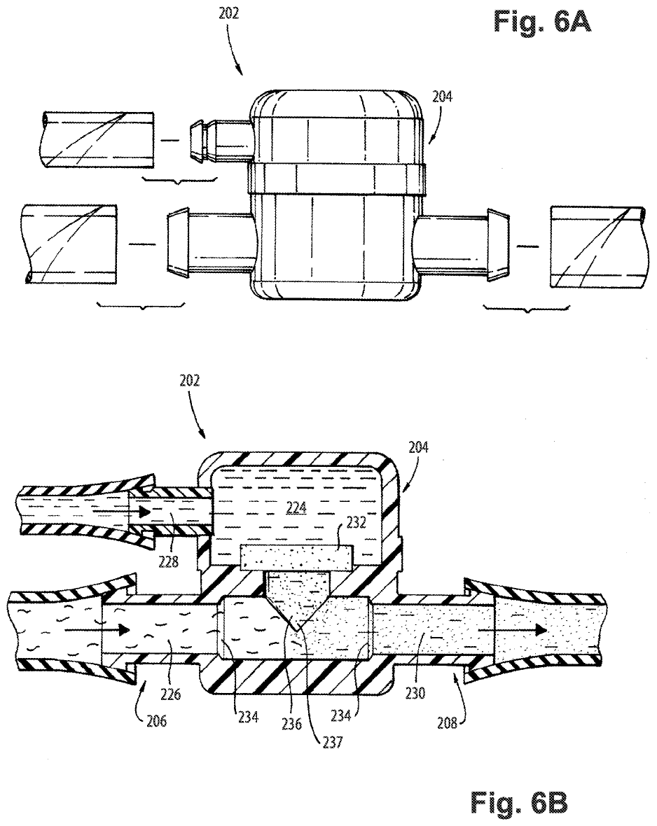

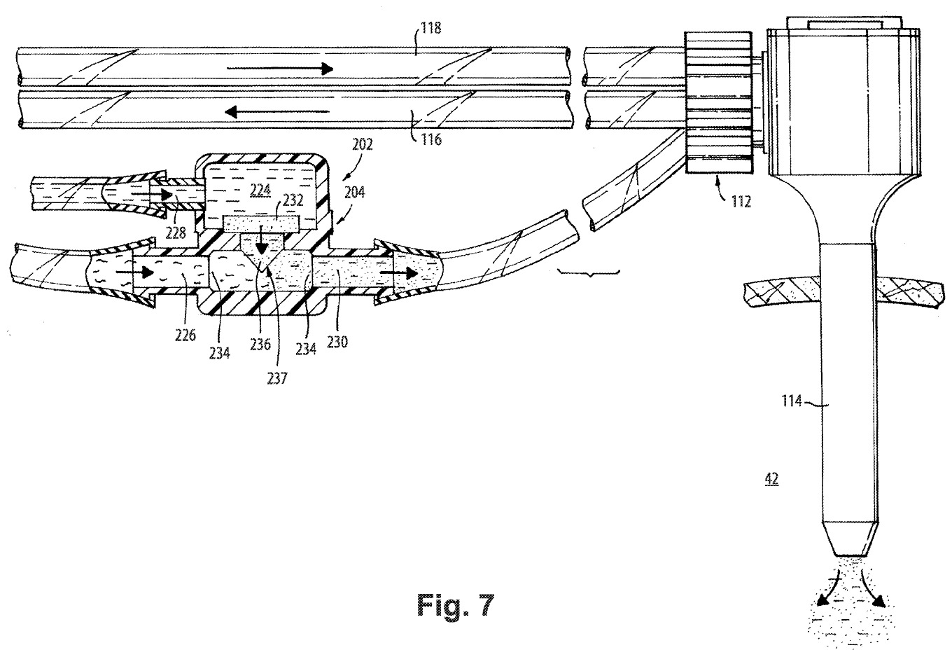

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1. Other embodiments and/or aspects of this disclosure are shown in FIGS. 2-10B. Certain embodiments described herein can be used to inject humidity into a flow of surgical gas.

There is illustrated in FIG. 1 a surgical gas delivery system 10 for use during endoscopic surgical procedures. The system 10 includes a device housing 12. The front face of the housing 12 has a capacitive or resistive touch screen 16 for presenting a graphical user interface (GUI) and a power switch 18 for turning the device on and off.

The front face of housing 12 further includes a filter cartridge interface 20 with a rotatable latch mechanism 22 configured to facilitate the secure engagement of a disposable filter cartridge 24 within the device housing 12. The rear face of the housing 12 includes a gas supply fitting 26 for connection with a source of compressed gas 28, a standard USB interface for service purposes, and a standard power connection (USB and power connection not shown).

In embodiments, the filter cartridge interface 20 is designed to recognize which type of filter 24 has been inserted into the housing. For example, it may recognize the proper position or orientation of the filter cartridge. It can also recognize if the inserted filter is specifically designed for use in the first mode of operation (i.e., the gaseous seal mode) or a filter specifically designed for use in the second mode of operation (i.e., insufflation and smoke evacuation mode). Other aspects of surgical gas delivery systems are described in U.S. Pat. No. 9,067,030, which is incorporated by reference herein in its entirety. Filter cartridge and/or tube set recognition can be accomplished, for example, using RFID techniques. The tube set and filter cartridge can be similar to that disclosed in U.S. Pat. No. 9,375,539 and U.S. Pat. No. 9,067,030, the entire contents of which are incorporated herein by reference.

In embodiments, a filtered tube set 36 described herein can be suitable for use with the surgical gas delivery device 10, e.g., as described above and as shown in FIGS. 1-4. As shown, the filtered tube set 36 includes a filter cartridge 24 having a housing 38 defining an inlet flow path 40 for receiving a flow of gas from a patient's abdominal cavity 42 and an outlet flow path 44 for delivering a flow of gas to the patient's abdominal cavity 42. In embodiments, the inlet flow path 40 of the filter cartridge and/or the outlet path 44 of the filter cartridge 24 can include one or more filter elements therein.

A first lumen 100 extends from a front end of the filter cartridge 24 and communicates with the outlet flow path 44 of the filter cartridge. A humidification device 102 is disposed in the first lumen 100 as an in-line humidification device 102 (e.g., any embodiment of a humidification device 102, 202 described hereinbelow). The in-line humidification device 102 has a housing 104, which includes an inlet fitting and an outlet fitting 108, the inlet fitting communicates with a distal end 101 of the first lumen 100.

A second lumen 110 extends from the outlet fitting 108 of the in-line humidification device 102 and communicates with the inlet flow path 40 of the filter cartridge 24. The second lumen 110 has a first connector 112 at a distal end 111 thereof for connecting to a surgical access device 114 (e.g., as shown and described in U.S. Pat. No. 7,854,724, the entire content of which is incorporated herein by reference). The second lumen 110 is configured to deliver a mixture of fluid vapor and surgical gas to a surgical cavity to increase a relative humidity of the surgical cavity 42.

A third lumen 116 having a connector (e.g., a common connector 112) at a distal end 117 thereof for connecting to the surgical access device 114 extends from the surgical access device 114 and communicates with the inlet flow path 42 of the filter cartridge 24. The third lumen is configured to deliver evacuated smoke from the surgical cavity to the cartridge. In embodiments, the system can further include the gas delivery system.

A fourth lumen 118 extends from the front end of the filter cartridge 24 for providing a jet flow to the surgical access device 114 to create an air seal at the surgical access device 114 to prevent egress of gas from the patient's abdominal cavity 42 through the surgical access device 114. The fourth lumen includes a third connector (e.g., common connector 112) at a distal end 119 thereof for connecting to the surgical access device 114. In certain embodiments, as shown, the second lumen 110, the third lumen 116, and the fourth lumen 118 can all be coupled the respective distal ends thereof such that the first connector, the second connector, and the third connector define a common connector 112 having respective flow paths therethrough. In certain embodiments, the common connector 112 can be a tri-lumen connector. A fifth lumen 120 connects a liquid source 122 to the in-line humidification device 102 at a second inlet fitting 122 to provide liquid to the in-line humidification device 102, as described further herein. From the in-line humidification device 102, the second lumen 110 is configured to deliver a mixture of fluid vapor and surgical gas (e.g., humidified surgical gas) to the surgical cavity 42 (e.g., through the surgical access device 114) to increase a relative humidity of the surgical cavity 42.

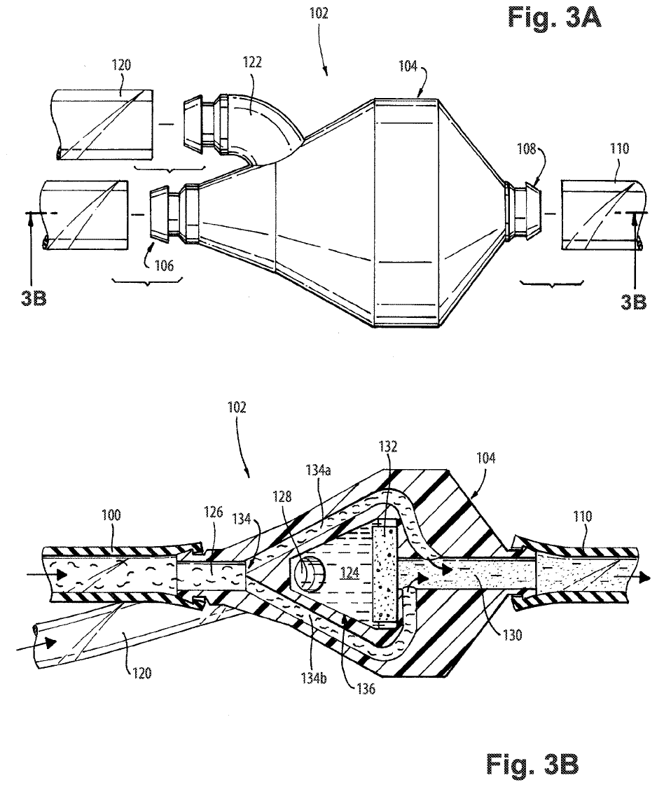

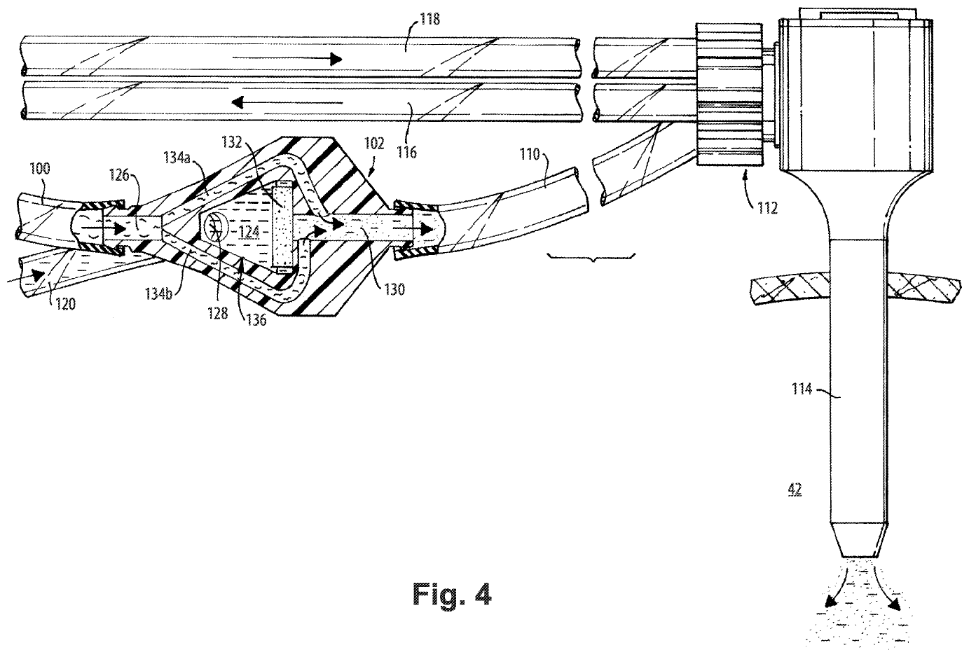

In accordance with at least one aspect of this disclosure, the in-line humidification device 102, in conjunction with the surgical gas delivery system 10 and filtered tube set 26 for a system for delivering humidified gas to the surgical access device 114. Referring now to FIGS. 3A-4, in which an embodiment of the humidification device 102 is shown, the housing 104 defines an interior humidification chamber 124. The interior humidification chamber 124 includes, a first inlet 126 for receiving a flow of gas, a second inlet 128 for receiving a flow of liquid, and an outlet 130 for delivering a flow of humidified gas to the surgical access device 114. A humidification mechanism 132 is disposed in the interior humidification chamber 124 configured to convert at least a portion of the flow of liquid to a flow of vapor for mixing with the flow of gas to form the flow of humidified gas that is ultimately delivered to the surgical access device 114. In embodiments, the humidification mechanism 132 is configured and adapted to disturb the flow of liquid within the interior humidification chamber 124 to convert the flow of liquid to the flow of vapor for mixing with the flow of gas, e.g., at the outlet 130 of the humidification chamber 124.

In embodiments, the humidification mechanism 132 is a nebulizer. In certain embodiments, the nebulizer can be or include a vibrating mesh nebulizer or an ultrasonic nebulizer. In certain embodiments, the mechanism 132 can include a jet nebulizer. In certain embodiments, the mechanism 132 can include a mechanical nozzle or any suitable means for forcing and/or pressurizing a liquid through a nozzle to create a vapor, aerosol or mist. In certain embodiments, the mechanism 132 can include a chemical reactor for chemically generating vapor, for example reacting hydrogen gas with oxygen gas to form water vapor.

In certain embodiments, as shown in FIGS. 3B and 4, in the interior humidification chamber 124, the first inlet 126 communicates with a branched flow path 134 formed within the housing 104 having a first branch 134a and a second branch 134b. The branched flow path 134 extends around a periphery 136 of the interior humidification chamber 124, and bypasses the interior humidification chamber 124, such that each of the first branch 134a and the second branch 134b join the outlet 130 separately and downstream of the interior humidification chamber 124. In such embodiments, the flow of gas and the flow of vapor are permitted to mix in the outlet 130 before being delivered to the surgical access device 114, which is shown in FIG. 4, for example.

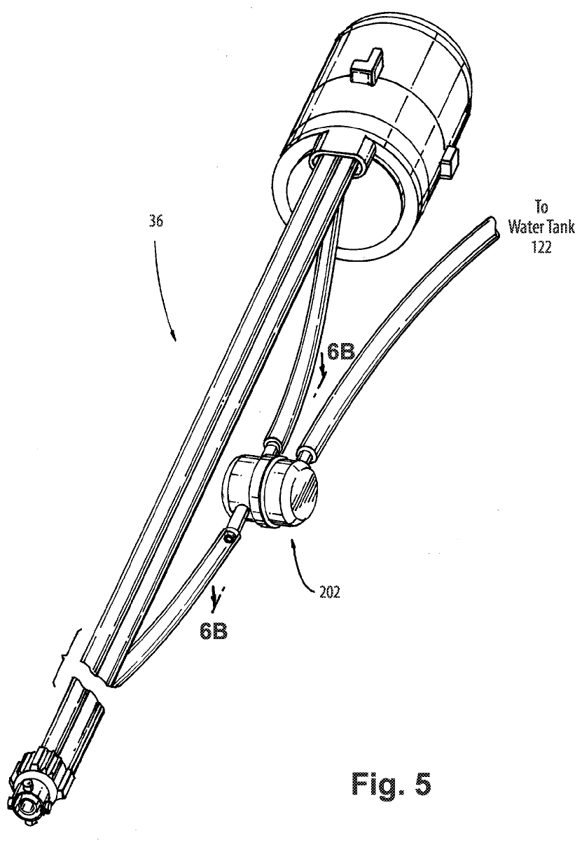

With reference now to FIGS. 5-7, another embodiment of a humidification device 202 is shown. The humidification device 202 can be similar to that of the humidification device 102, for example humidification device 202 can have similar components and features with respect to humidification device 102. For brevity, the description of common elements that have been described above for humidification device 102 are not repeated with respect to humidification device 202 as shown in FIGS. 5-7.

The humidification device 202 can be an in-line humidification device 202 within the filtered tube set 36 similar to that described above with respect to in-line humidification device 102 and shown in FIG. 2. For humidification device 202, the interior humidification chamber 224 includes a first inlet 226 communicating with a first flow path 234 formed within the housing 204. The first flow path 234 bypasses the interior humidification chamber 224, and the second inlet 228 communicates directly with the interior humidification chamber 224 within the housing 204. The first flow path 234 and an outlet 236 of the humidification chamber 224 meet at a T-junction 237 downstream of the humidification mechanism 232 and upstream of the outlet 230. Here, the flow of gas and the flow of vapor mix in the T-junction 237, downstream of the interior humidification chamber 224.

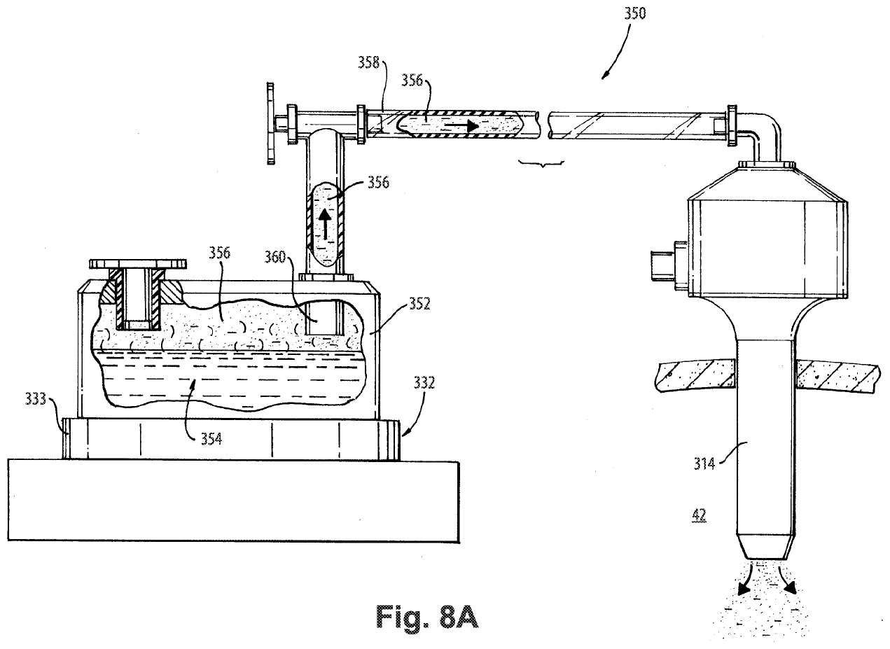

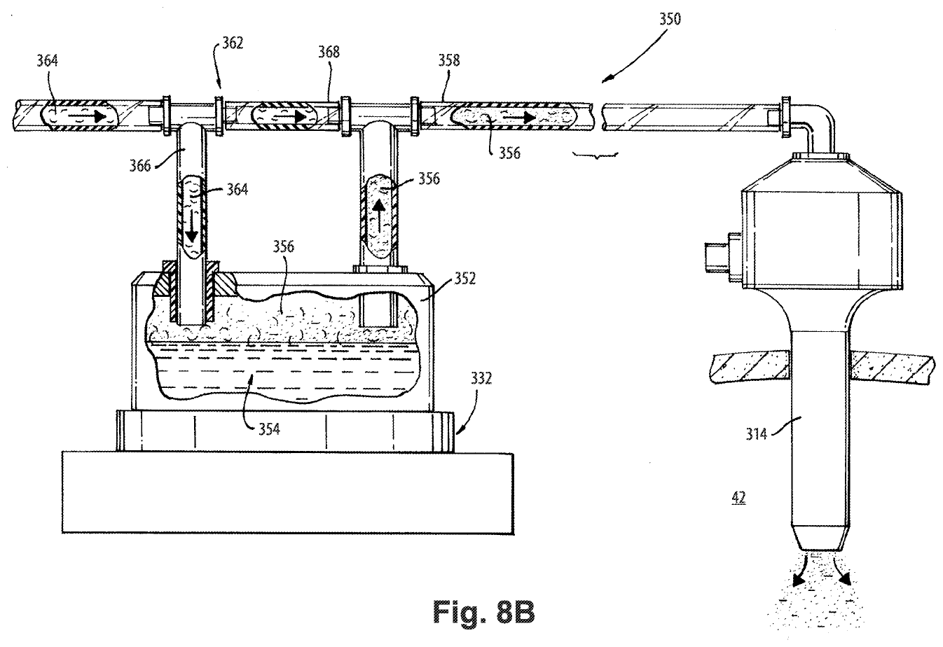

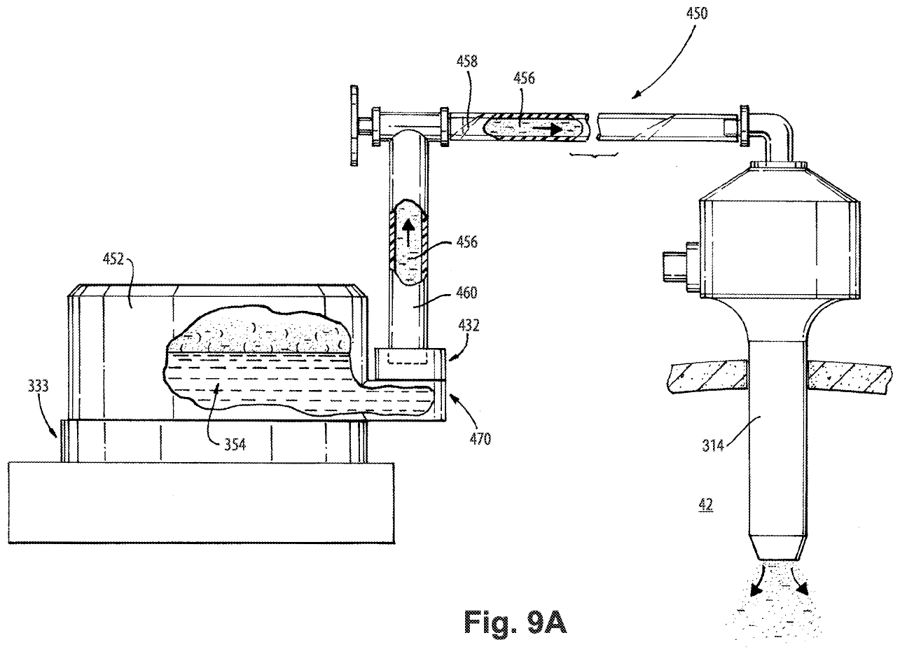

With reference now to FIGS. 8A and 8B, in accordance with at least one aspect of this disclosure, a system 350 for introducing humidity into the surgical cavity 42 includes a surgical access device 314 configured to deliver humidity to the surgical cavity 42 to increase a relative humidity of the surgical cavity 42. The system 350 includes a reservoir 352 configured to retain a fluid 354 (e.g., at least a liquid) therein and a humidification mechanism 332 is operatively connected to at least the reservoir 352 configured and adapted to disturb the liquid 354 in the reservoir 352 to convert at least a portion of the liquid 354 to a vapor state 356 to produce humidity within the reservoir 352. A first lumen 358 is fluidly connected between the reservoir 352 and the surgical access device 314 to provide the vapor 356 to the surgical access device 314 to deliver the humidity to the surgical cavity 42 through the surgical access device 314. In embodiments, the liquid 354 can be an aqueous solution, and in certain embodiments, the aqueous solution can be a saline solution.

In embodiments, e.g., as shown in FIGS. 8A and 8B, the humidification mechanism 332 can include a heating element 333 in thermal communication with the reservoir 352 configured and adapted to vaporize the liquid 354 within the reservoir to produce the vapor 356. An inlet 360 of the first lumen 358 is in fluid communication with the vapor 356 in the reservoir 352 to passively provide the vapor 356 to the surgical access device 314. In certain embodiments, as shown in FIG. 8B, a second lumen 362 can be fluid communication with a gas source (e.g., CO2) configured to deliver a flow of pressurized gas 364 to the first lumen 358 to provide a back pressure on the reservoir 352 and/or humidification mechanism 332. Here, a first portion 366 of the second lumen 362 is in fluid communication with the reservoir 352 for delivering the flow of pressurized gas 364 to the reservoir 352. The second lumen 362 includes a bypass portion 368 in fluid communication with the first lumen 358 downstream of the reservoir 352 (relative to the flow through the reservoir) and upstream of the surgical access device 314.

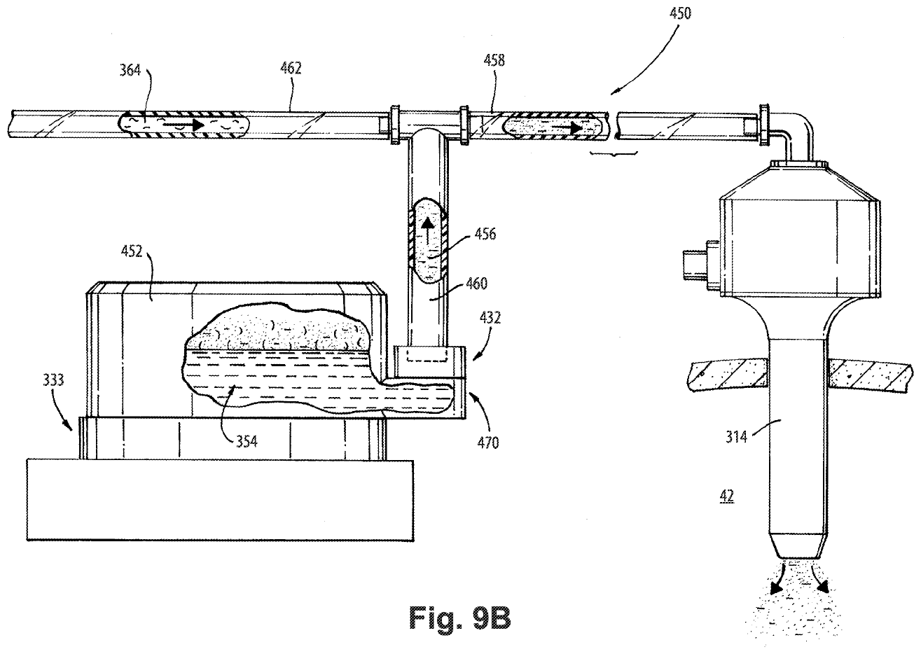

With reference now to FIGS. 9A and 9B, another embodiment of a system 450 is shown. The system 450 can be similar to that of the system 350, for example system 450 can have similar components and features with respect to system 350. For brevity, the description of common elements that have been described above for system 350 are not repeated with respect to system 450 as shown in FIGS. 9A and 9B.

In system 450, the humidification mechanism 452 includes nebulizer disposed at the inlet 460 of the first lumen 458 configured and adapted to convert the liquid 354 at an outlet 470 of the reservoir 452 and the inlet 460 of the first lumen 458. In embodiments, the nebulizer 432 can be or include a vibrating mesh nebulizer, or an ultrasonic nebulizer. In embodiments, as shown in FIG. 9B, a second lumen 462 can be in fluid communication with a gas source configured to deliver a flow of pressurized gas 364 to the first lumen 458 to provide a back pressure on the humidification mechanism 432. Here, the second lumen 462 can intersect the first lumen 458 downstream of the reservoir 452 and upstream of the surgical access device 314, e.g., relative to the flow of the vapor 456 through the first lumen 458. In embodiments, the heating element 333 can still be in thermal communication with the reservoir configured to heat the liquid 354 within the reservoir 352 such that a temperature of the humidity 456 delivered to the surgical cavity is greater than a temperature of the pressurized gas 364 in the second lumen 462.

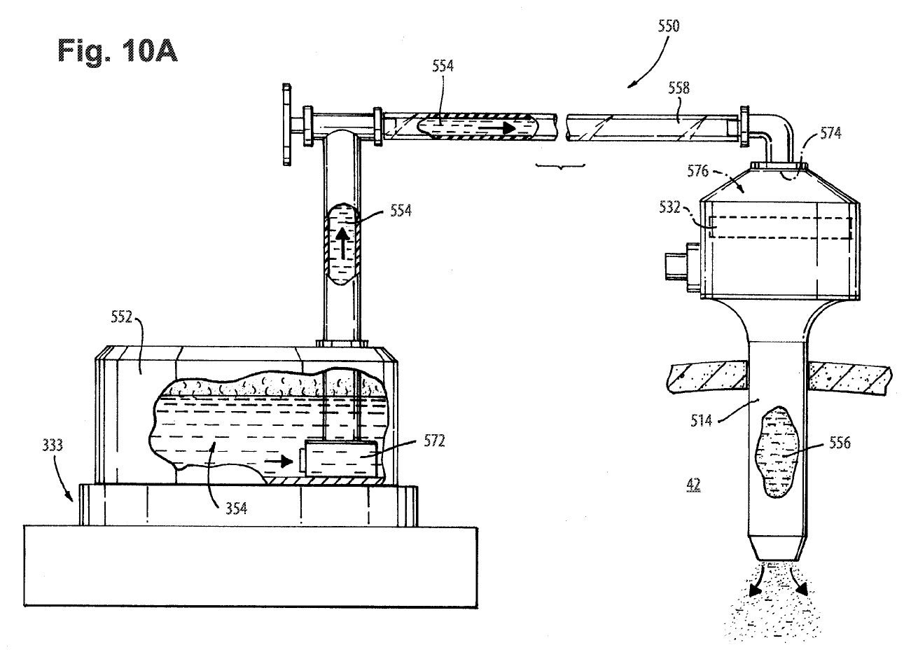

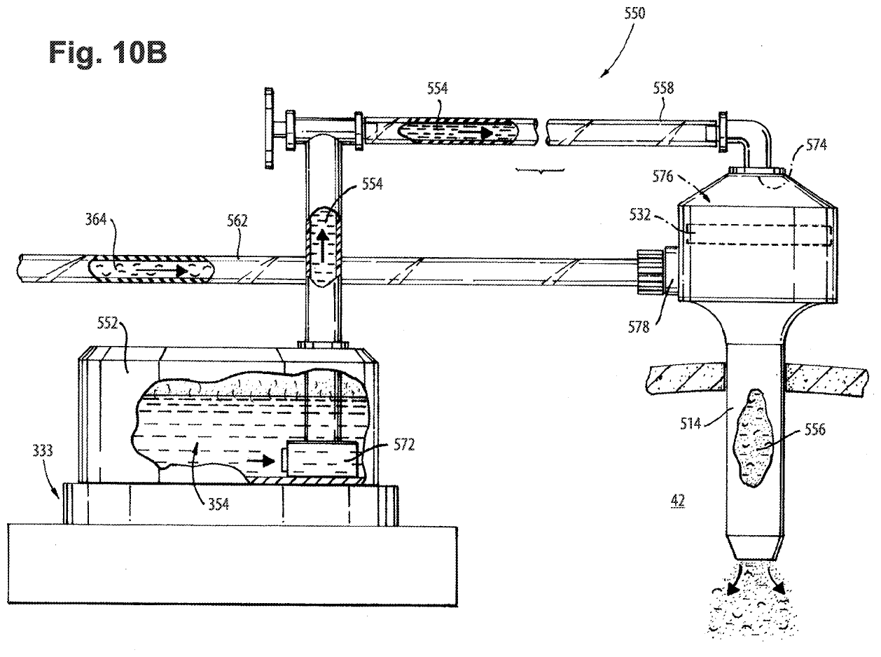

With reference now to FIGS. 10A and 10B, another embodiment of a system 550 is shown. The system 550 can be similar to that of the system 450, for example system 550 can have similar components and features with respect to system 450. For brevity, the description of common elements that have been described above for system 450 are not repeated with respect to system 550 as shown in FIGS. 10A and 10B.

In certain embodiments, a pump 572 is disposed in the reservoir 552 configured and adapted to pump the liquid 354 to the surgical access device 514 at a pressure, and the humidification mechanism 532 is operatively connected to the surgical access device 514 configured to convert the pressurized liquid 554 a vapor 556 to produce the humidity. The humidification mechanism 532 can be or include a nebulizer disposed between an outlet 574 of the first lumen 558 and within an inlet 576 of the surgical access device 514 configured and adapted to vaporize the pressurized liquid 554 in the first lumen 558 within the inlet 576 of the surgical access device 514. In certain embodiments, as shown in FIG. 10B, a second lumen 562 can be in fluid communication with a gas source configured to deliver a flow of pressurized gas 364 to the surgical access device 514 to provide a back pressure on the humidification mechanism 532. Here, an outlet 578 of the second lumen 562 can intersect at the surgical access 514 device downstream of the outlet 574 of the first lumen 558 and downstream of the humidification mechanism 532. In embodiments, the heating element 333 can be in thermal communication with the reservoir 552 configured to heat the liquid 354 within the reservoir such that a temperature of the humidity delivered 556 to the surgical cavity 42 is greater than a temperature of the pressurized gas 364 in the second lumen 562.

In embodiments, the surgical access device 314, 514 is a first surgical access device, and the system 351, 450, 550 can include a surgical gas delivery system configured and adapted to deliver a flow of insufflation gas to the surgical cavity through a second surgical access device (e.g., similar to that described above with respect to access device 114). In certain embodiments, the surgical access device 314, 514 can be a single surgical access device for delivering humidity and surgical gas, where the surgical gas delivery system configured and adapted to deliver the flow of gas to the surgical cavity through the surgical access device 314, 514. The surgical gas can include insufflation gas (e.g., CO2).

In at least one embodiment of the system, the surgical access device 314, 514 can be a first surgical access device, and a surgical gas delivery system can be included configured to deliver a flow of insufflation gas to the surgical cavity through a second surgical access device. The surgical gas delivery system can be the same or similar to that of U.S. Pat. No. 9,067,030, and similar to that described above. The surgical gas can include insufflation gas, such as CO₂. In embodiments, the fluid (e.g., the fluid held within the reservoir for generating the humidity) can include an aqueous solution, such as a saline solution, for example.

Embodiments can utilize heated water as a vapor source for humidification and a heater can be used as the mechanism for generating vapor. Water vapor can travel from a reservoir along a lumen to the surgical cavity. Embodiments can use heated water with pressurized CO₂. Pressurized CO₂ forces water vapor and CO₂ mix while travelling along the lumen to the surgical cavity.

In embodiments, a nebulizer can be used to generate vapor for humidification using a water/saline reservoir. The nebulizer can include one or more of a vibrating mesh and/or ultrasonic nebulizer and/or a jet nebulizer. The nebulizer can sit in the water/saline reservoir and create a cold humidification. As used herein, cold refers to a temperature that is not actively heated. The temperature of the humidified gas may be warmer than that of the gas in the gas source, however it is colder than heated insufflation gas. The water/saline vapor travels along the lumen to the surgical cavity. Embodiments can include a vibrating mesh nebulizer with positive water/saline pressure on the back of the nebulizer. In embodiments, the nebulizer can sit at an opening of a surgical access device with positive solution pressure on back of nebulizer. In this case, the water/saline solution vapor is forced down surgical access device and into the cavity.

In certain embodiments, pressurized CO₂ can be used to provide additional force to push the vapor into the surgical cavity, to dilute the vapor, or to provide a vortex around an inner diameter of the access device to prevent water build up.

Ultrasonic nebulizers can operate at a high frequency, e.g., about 1-5 MHz, and generates vapor by forming a standing wave, capillary wave, that generates a geyser of droplets that break free as fine aerosol particles above the transducer. Ultrasonic nebulizers can generate lower droplet velocity and more heat. Vibrating mesh or plate nebulizers can operate at a lower frequency, e.g., 110 kHz, and forces a solution through holes in a plate to create a micro pump that generates the vapor. Vibrating mesh or plate nebulizers can generate greater droplet velocity, less heat, and can be used with medications or pharmaceuticals. here remains a need in the art for improved systems for providing humidity to insufflation gas before supplying the insufflation gas to the surgical cavity. This disclosure provides a solution for this need.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. A system for introducing humidified gas to a surgical access device, comprising:
a humidification device including a housing defining an interior humidification chamber having:
a first inlet for receiving a flow of gas;
second inlet for receiving a flow of liquid;
a humidification mechanism configured to convert at least a portion of the flow of liquid to a flow of vapor for mixing with the flow of gas to form a flow of humidified gas; and
an outlet for delivering the flow of humidified gas to the surgical access device.

2. The system of claim 1, wherein the humidification mechanism is configured and adapted to disturb the flow of liquid within the interior humidification chamber to convert the flow of liquid to the flow of vapor for mixing with the flow of gas.

3. The system of claim 2, wherein the humidification mechanism is a nebulizer.

4. The system of claim 3, wherein the nebulizer includes a vibrating mesh nebulizer or an ultrasonic nebulizer.

5. The system of claim 2, wherein the first inlet communicates with a branched flow path formed within the housing having a first branch and a second branch, wherein the branched flow path extends around a periphery of the interior humidification chamber, bypassing the interior humidification chamber, such that each of the first branch and the second branch join the outlet separately and downstream of the interior humidification chamber, wherein the flow of gas and the flow of vapor mix in the outlet.

6. The system of claim 2, wherein the first inlet communicates with a first flow path formed within the housing, wherein the first flow path bypasses the interior humidification chamber, and wherein the second inlet communicates directly with the interior humidification chamber within the housing, wherein the first flow path and the second flow path meet at a T-junction upstream of the outlet, wherein the flow of gas and the flow of vapor mix in the T-junction, downstream of the interior humidification chamber.

7. The system of claim 1, wherein the housing further includes a first inlet fitting, a second inlet fitting and an outlet fitting, and further comprising:
a first lumen configured to connect to the first inlet fitting to deliver the flow of gas to the interior humidification chamber from a gas source;
a second lumen configured to connect to the second inlet fitting to deliver the flow of liquid to the interior humidification chamber from a liquid source; and a
third lumen configured to connect to the outlet fitting to deliver the flow of humidified gas from the interior humidification chamber to the surgical access device.

8. The system of claim 1, further comprising,
a tube set for use with a surgical gas delivery device, comprising:
a filter cartridge having a housing defining a inlet flow path for receiving a flow of gas from a patient's abdominal cavity and an outlet flow path for delivering a flow of gas to the patient's abdominal cavity;
a first lumen extending from a front end of the cartridge and communicating with the outlet flow path of the filter cartridge;
wherein the humidification device is an in-line humidification device, the in-line humidification device including an inlet fitting and an outlet fitting, wherein the inlet fitting communicates with a distal end of the first lumen;
a second lumen extending from the outlet fitting of the in-line humidification device and communicating with the inlet flow path, wherein the second lumen has a first connector at a distal end thereof for connecting to a surgical access device; and
a third lumen having a second connector at a distal end thereof for connecting to the surgical access device and extending from the surgical access device and communicating with the inlet flow path of the filter cartridge.

9. The system of claim 8, wherein the inlet flow path of the filter cartridge and/or the outlet path of the filter cartridge includes one or more filter elements therein.

10. The system of claim 9, further comprising a fourth lumen extending from the front end of the filter cartridge for providing a jet flow to the surgical access device to create an air seal at the surgical access device to prevent egress of gas from the patient's abdominal cavity through the surgical access device.

11. The system of claim 10, wherein the fourth lumen includes a third connector at a distal end thereof for connecting to the surgical access device.

12. The system of claim 11, wherein the second lumen, the third lumen, and the fourth lumens are coupled at distal ends thereof and wherein the first connector, the second connector, and the third connector define a common connector having respective flow paths therethrough.

13. The system of claim 8, wherein the second lumen is configured to deliver a mixture of fluid vapor and surgical gas to a surgical cavity to increase a relative humidity of the surgical cavity, and wherein third lumen is configured to deliver evacuated smoke from the surgical cavity to the cartridge.

14. A system, comprising:
a surgical access device configured to deliver humidity to a surgical cavity to increase a relative humidity of the surgical cavity;
a reservoir configured to retain at least a liquid therein;
a humidification mechanism operatively connected to at least the reservoir configured and adapted to disturb the liquid in the reservoir to convert at least a portion of the liquid to a vapor state to produce humidity within the reservoir; and
a first lumen fluidly connected between the reservoir and the surgical access device to provide the vapor to the surgical access device to deliver the humidity to the surgical cavity through the surgical access device.

15. The system of claim 14, wherein the humidification mechanism includes a heating element in thermal communication with the reservoir configured and adapted to vaporize the liquid within the reservoir to produce the vapor, wherein an inlet of the first lumen is in fluid communication with the vapor in the reservoir.

16. The system of claim 15, wherein the humidification mechanism includes nebulizer disposed at an inlet of the first lumen configured and adapted to convert the liquid at an outlet of the reservoir and the inlet of the first lumen.

17. The system of claim 16, wherein the nebulizer includes a vibrating mesh nebulizer, or an ultrasonic nebulizer.

18. The system of claim 14, further comprising a pump disposed in the reservoir configured and adapted to pump the liquid to the surgical access device at a pressure, and further comprising a second humidification mechanism operatively connected to at least the surgical access device configured to convert the pressurized liquid a vapor to produce the humidity, wherein the first humidification mechanism includes a heating element in thermal communication with the reservoir configured and adapted to vaporize the liquid within the reservoir, wherein the first lumen is in fluid communication with the fluid vapor in the reservoir, and wherein the second humidification mechanism includes a nebulizer disposed between an outlet of the first lumen and within an inlet of the surgical access device configured and adapted to vaporize the pressurized liquid in the first lumen within the inlet of the surgical access device.

19. The system of claim 14, further comprising a second lumen in fluid communication with a gas source configured to deliver a flow of pressurized gas to the first lumen to provide a back pressure on the humidification mechanism, wherein the second lumen intersects the first lumen downstream of the reservoir and upstream of the surgical access device.

20. The system of claim 19, further comprising a heating element in thermal communication with the reservoir configured to heat the liquid within the reservoir such that a temperature of the humidity delivered to the surgical cavity is greater than a temperature of the pressurized gas in the second lumen.
